(19) United States
(12) Patent Application Publication
(10) Pub. No.: US 2021/0356345 A1
(43) Pub. Date: Nov. 18, 2021

(54) PRESSURE VISUALIZATION DEVICE, MANUFACTURING METHOD THEREOF, AND DETECTION DEVICE

(71) Applicants: Beijing BOE Display Technology Co., Ltd., Beijing (CN); BOE Technology Group Co., Ltd., Beijing (CN)

(72) Inventor: Yanqiu LI, Beijing (CN)

(21) Appl. No.: 16/329,325

(22) PCT Filed: Oct. 11, 2018
(86) PCT No.: PCT/CN2018/109851

§ 371 Date: Feb. 28, 2019

(30) Foreign Application Priority Data
Mar. 23, 2018 (CN) 201810247405.3

(51) Int. Cl. G01L 19/08; G02F 1/155; G02F 1/163; G02F 1/1524; H01L 41/08; H01L 41/113; H01L 41/187; H01L 41/22; G01L 9/08; A61B 5/022; A61B 5/318; A61B 5/00

(52) U.S. Cl. CPC G01L 19/08; G02F 1/155; G02F 1/163; G02F 1/1524; H01L 41/081; H01L 41/082; A61B 2562/0247; H01L 41/187; H01L 41/22; G01L 9/08; A61B 5/022; A61B 5/318; A61B 5/6801; H01L 41/1132

(57) ABSTRACT

A pressure visualization device includes a flexible substrate, a piezoelectric module and an electrochromic module disposed on a surface of the flexible substrate and adjacent to each other, a first attachment layer on a surface of the piezoelectric module facing away from the flexible substrate, and a second attachment layer on the other surface of the flexible substrate; the piezoelectric module includes a plurality of piezoelectric units each including a first electrode, a second electrode, and a piezoelectric layer between the first electrode and the second electrode; the electrochromic module includes a plurality of electrochromic units each including a third electrode, a fourth electrode, an electrochromic layer between the third electrode and the fourth electrode; wherein the second electrode is electrically connected to the third electrode, and the fourth electrode is a transparent electrode.

### CROSS REFERENCE TO RELATED APPLICATIONS

The present application is based upon International Application No. PCT/CN2018/109851, filed on Oct. 11, 2018, which claims priority of Chinese patent application No. 201810247405.3, filed on Mar. 23, 2018, the entire disclosure of which is hereby incorporated by reference as a part of the present application.

### TECHNICAL FIELD

The present disclosure relates to the field of detection technology, and more particularly to a pressure visualization device, a manufacturing method thereof, and a detection device.

### BACKGROUND

A traditional pressure detection system primarily includes a pressure sensor and a display. In the detection process, signals detected by the pressure sensor is recorded in real time, and then a pressure graph is drawn according to the recorded signals, and finally the pressure graph drawn is displayed by the display to reflect the pressure change process.

It should be noted that the information disclosed in the Background section above is only for enhancement of understanding of the background of the present disclosure, and thus may include information that does not constitute prior art known to those of ordinary skill in the art.

### SUMMARY

An object of the present disclosure is to provide a pressure visualization device, a manufacturing method thereof, and a detection device.

Other features and advantages of the present disclosure will be apparent from the following detailed description, or learned in part from practice of the present disclosure.

According to one aspect of the present disclosure, there is provided a pressure visualization device, including a flexible substrate, a piezoelectric module and an electrochromic module on a surface of the flexible substrate;

wherein the piezoelectric module includes a plurality of piezoelectric units each including a first electrode close to the flexible substrate, a second electrode away from the flexible substrate, and a piezoelectric layer between the first electrode and the second electrode;

the electrochromic module includes a plurality of electrochromic units each including a third electrode close to the flexible substrate, a fourth electrode away from the flexible substrate, and an electrochromic layer between the third electrode and the fourth electrode; and

the second electrode is electrically connected to the third electrode, and the fourth electrode is a transparent electrode.

In an exemplary embodiment of the present disclosure, a sum of an occupied area of the piezoelectric module on the flexible substrate and an occupied area of the electrochromic module on the flexible substrate is equal to a surface area of the flexible substrate.

In an exemplary embodiment of the present disclosure, the first attachment layer and the second attachment layer each includes a hydrogel.

In an exemplary embodiment of the present disclosure, the pressure visualization device further includes a first attachment layer on a surface of the piezoelectric module facing away from the flexible substrate, a second attachment layer on the other surface of the flexible substrate, and the hydrogel is obtained by physically cross linking amorphous calcium carbonate nanoparticles, polyacrylic acid, and sodium alginate.

In an exemplary embodiment of the present disclosure, the second electrode is disposed in the same layer and has the same material as the third electrode.

In an exemplary embodiment of the present disclosure, the pressure visualization device further includes a protective layer on a side of the electrochromic module facing away from the flexible substrate.

In an exemplary embodiment of the present disclosure, the protective layer includes a transparent resin layer and the material of the transparent resin layer includes polydimethylsiloxane.

In an exemplary embodiment of the present disclosure, the piezoelectric module further includes a conductive layer between the first electrode and the piezoelectric layer, and the piezoelectric layer includes zinc oxide nanowires.

In an exemplary embodiment of the present disclosure, the electrochromic layer includes a tungsten trioxide pattern layer, and a current amplification circuit is provided in the tungsten trioxide pattern layer.

According to one aspect of the present disclosure, there is provided a manufacturing method of a pressure visualization device, including:

forming a flexible substrate layer, a first electrode, and a resin layer sequentially over a glass substrate, and patterning the resin layer to obtain a slot in a first region and a resin retaining layer in a second region, the first region and the second region being disposed adjacent to each other;

forming a piezoelectric layer and a second electrode sequentially in the slot;

forming a third electrode, an electrochromic layer, and a fourth electrode sequentially over the resin retaining layer, the third electrode being electrically connected to the second electrode, and the fourth electrode being a transparent electrode; and

peeling off the flexible substrate layer from an interface of the glass substrate and the flexible substrate layer;

wherein the first region is for providing a piezoelectric module, the piezoelectric module includes a plurality of piezoelectric units each including the first electrode, the second electrode, and the piezoelectric layer; the second region is for providing an electrochromic module, and the electrochromic module includes a plurality of electrochromic units each including the third electrode, the fourth electrode, and the electrochromic layer.

In an exemplary embodiment of the present disclosure, a sum of an area of the first region and an area of the second region is equal to a surface area of the flexible substrate layer.

In an exemplary embodiment of the present disclosure, the manufacturing method further includes:

after sequentially forming the piezoelectric layer and the second electrode in the slot, forming a first attachment layer over the second electrode; and

after peeling off the flexible substrate layer from the interface between the glass substrate and the flexible substrate layer, forming a second attachment layer on the peeling surface of the flexible substrate layer,

wherein the materials of the first attachment layer and the second attachment layer all include a hydrogel.

In an exemplary embodiment of the present disclosure, the hydrogel is obtained by physically cross linking amorphous calcium carbonate nanoparticles, polyacrylic acid, and sodium alginate.

In an exemplary embodiment of the present disclosure, the second electrode and the third electrode are prepared by performing the same patterning process on the same film layer; and

before forming the second electrode and the third electrode, the manufacturing method further includes:

patterning the resin retaining layer to obtain a recess for forming the third electrode.

In an exemplary embodiment of the present disclosure, the manufacturing method further includes: forming a protective layer over the fourth electrode.

In an exemplary embodiment of the present disclosure, the protective layer includes a transparent resin layer, and the material of the transparent resin layer includes polydimethylsiloxane.

In an exemplary embodiment of the present disclosure, the piezoelectric module further includes a conductive layer formed between the first electrode and the piezoelectric layer, and the piezoelectric layer includes zinc oxide nanowires.

In an exemplary embodiment of the present disclosure, the electrochromic layer includes a tungsten trioxide pattern layer, and a current amplifying circuit is further formed in the tungsten trioxide pattern layer.

According to one aspect of the present disclosure, there is provided a detection device, including the pressure visualization device described above.

In an exemplary embodiment of the present disclosure, the detection device includes a sphygmomanometer and/or an electrocardiograph.

In an exemplary embodiment of the present disclosure, the detection device is a wearable device.

It should be understood that the above general description and the following detailed description are intended to be exemplary and illustrative, rather than to limit the present disclosure. This section provides an overview of various implementations or examples of the techniques described in the present disclosure, and is not a comprehensive disclosure of the full scope or all features of the disclosed technology.

### BRIEF DESCRIPTION OF THE DRAWINGS

The accompanying drawings, which are incorporated in and constitute a part of this specification, illustrate embodiments consistent with the disclosure and, together with the description, serve to explain the principles of the disclosure. It is apparent that the drawings in the following description are only some of the embodiments of the present disclosure, and other drawings may be obtained from these drawings by those skilled in the art without paying creative effort.

FIG. 1 is a schematic block diagram showing a structure of a pressure visualization device according to an exemplary embodiment of the present disclosure;

FIG. 2 is a schematic diagram showing a use state of a pressure visualization device according to an exemplary embodiment of the present disclosure;

FIG. 3 is a schematic diagram showing another use state of the pressure visualization device according to the exemplary embodiment of the present disclosure;

FIG. 4 schematically illustrates a capacitance-pressure response graph of a hydrogel pressure sensor according to an exemplary embodiment of the present disclosure;

FIG. 5 schematically illustrates a capacitance-pressure cycle graph of a hydrogel pressure sensor according to an exemplary embodiment of the present disclosure;

FIG. 6 schematically illustrates a real-time capacitance response graph of a hydrogel pressure sensor detecting water droplets according to an exemplary embodiment of the present disclosure;

FIG. 7 schematically illustrates a plurality of performance graphs of an electrochromic module 30 of a tungsten trioxide electrochromic layer 303 according to an exemplary embodiment of the present disclosure;

FIG. 8 is a diagram schematically illustrating a distribution effect of piezoelectric units and pattern imprints displayed by an electrochromic module under different pressures according to an exemplary embodiment of the present disclosure;

FIG. 9 is a diagram schematically showing a linear relationship between an enhancement ratio of a pattern imprint and an applied pressure according to an exemplary embodiment of the present disclosure;

FIG. 10 is a flow chart schematically showing a manufacturing method of a pressure visualization device according to an exemplary embodiment of the present disclosure;

FIGS. 11 to 14 are schematic diagrams showing a manufacturing process of a pressure visualization device according to an exemplary embodiment of the present disclosure; and

FIGS. 15 to 18 are schematic diagrams showing details of a manufacturing process of a pressure visualization device according to an exemplary embodiment of the present disclosure.

### DETAILED DESCRIPTION

Exemplary embodiments will now be described more fully with reference to the accompanying drawings. However, the exemplary embodiments can be embodied in a variety of forms and should not be construed as being limited to the examples set forth herein. Rather, these embodiments are provided so that this disclosure will be more complete and thorough, and to fully convey the concept of the exemplary embodiments to those skilled in the art. The described features, structures, or characteristics may be combined in any suitable manner in one or more embodiments.

In addition, the drawings are merely schematic representations of the present disclosure and are not necessarily drawn to scale. The same reference numerals in the drawings denote the same or similar parts, and the repeated description thereof will be omitted. Some of the block diagrams shown in the figures are functional entities and do not necessarily have to physically or logically correspond to separate entities. These functional entities may be implemented in software, or implemented in one or more hardware modules or integrated circuits, or implemented in different network and/or processor devices and/or microcontroller devices.

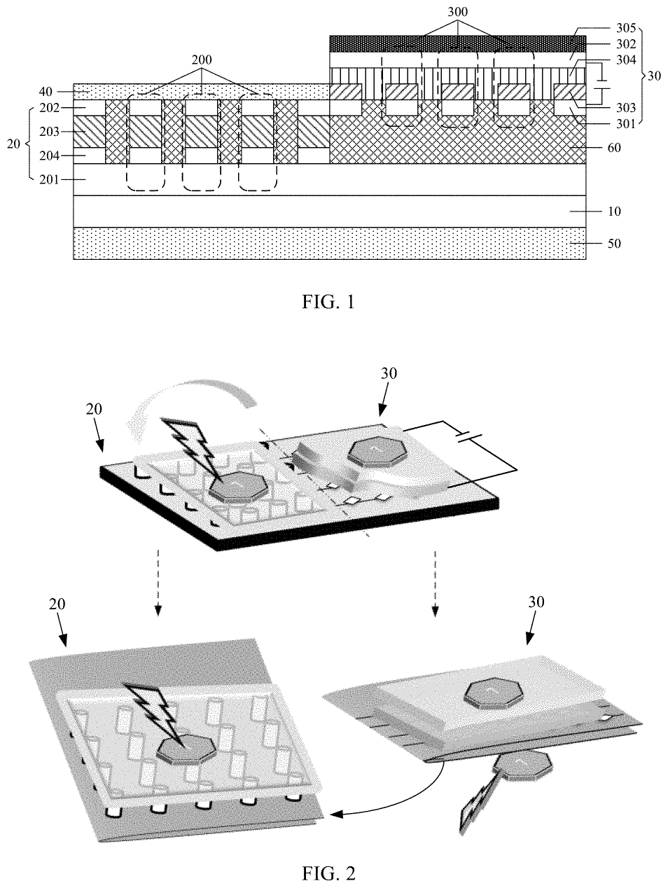

The present example embodiment provides a pressure visualization device that can be used in the field of medical testing such as electrocardiogram monitoring or blood pressure monitoring. As shown in FIG. 1, the pressure visualization device may include a flexible substrate 10, a piezoelectric module 20 and an electrochromic module 30 disposed on a surface of the flexible substrate 10 and disposed adjacent to each other, a first attachment layer 40 on one side of the piezoelectric module 20 away from the flexible substrate 10, and a second attachment layer 50 on the other surface of the flexible substrate 10. The first attachment layer 40 and the second attachment layer 50 may be attached to the surface of the test subject for sensing the pressure change on the surface of the test subject.

The piezoelectric module 20 may include a plurality of piezoelectric units 200. Each of the piezoelectric units 200 may include a first electrode 201 on a side close to the flexible substrate 10, a second electrode 202 on a side away from the flexible substrate 10, and a piezoelectric layer 203 between the first electrode 201 and the second electrode 202. The first electrode 201 may be laid on the entire surface of the flexible substrate 10 or only in a region corresponding to the piezoelectric module 20. The second electrode 202 may include a plurality of independent electrode blocks, and the piezoelectric layer 203 may correspondingly include a plurality of independent piezoelectric layer units. Then, the first electrode 201, the plurality of electrode blocks of the second electrode 202, and the plurality of piezoelectric layer units of the piezoelectric layer 203 can form a plurality of piezoelectric units 200 of the piezoelectric module 20.

The electrochromic module 30 may include a plurality of electrochromic units 300. Each of the electrochromic units 300 may include a third electrode 301 on a side close to the flexible substrate 10, a fourth electrode 302 on a side away from the flexible substrate 10, and an electrochromic layer 303 and an ion transport layer 304 between the third electrode 301 and the fourth electrode 302. The third electrode 301 may include a plurality of independent electrode blocks, and the fourth electrode 302 may be a transparent plate electrode or a plurality of electrically connected transparent block electrodes. The electrochromic layer 303 and the ion transport layer 304 may correspondingly include a plurality of independent electrochromic layer units and a plurality of independent ion transport layer units respectively. Then, the plurality of electrode blocks of the third electrode 301, the fourth electrode 302, and the plurality of electrochromic layer units of the electrochromic layer 303 and the plurality of ion transport layer units of the ion transport layer 304 can form a plurality of electrochromic units 300 of the electrochromic module 30. It should be understood that the electrochromic layer 303 and the ion transport layer 304 may constitute the electrochromic unit according to the present embodiment. However, the present disclosure is not limited thereto, and other electrochromic units, for example, electrochromic units omitting the ion transport layer or based on others principle or structural can also be applied to the present disclosure.

It should be noted that the electrical connection between the second electrode 202 and the third electrode 301 may be maintained to facilitate the transmission of electrical signals generated in the piezoelectric module 20 to the electrochromic module 30.

The pressure visualization device provided by the exemplary embodiment of the present disclosure can convert the pressure signal sensed by the piezoelectric module 20 into an electrical signal, and then excite the electrochromic module 30 to emit light and change color under the control of the electrical signal. It can not only realize detection of pressure, but also the detected pressure can be visually displayed instantly. It can be seen that the pressure visualization device can instantly display the pressure signal graph without an external display device, thereby realizing the visualization of the pressure signal. In addition, since the piezoelectric module 20 and the electrochromic module 30 are integrally disposed on the flexible substrate 10, the pressure visualization device also has the advantage of being small and portable.

In the present exemplary embodiment, the sum of the occupied area of the piezoelectric module 20 on the flexible substrate 10 and the occupied area of the electrochromic module 30 on the flexible substrate 10 may be equal to the surface area of the flexible substrate 10. The area of the piezoelectric module 20 and the area of the electrochromic module 30 can be, for example, exactly the same.

Based on the above structure, in consideration of simplification of the manufacturing process, the second electrode 202 and the third electrode 301 may be disposed in the same layer and have the same material. For example, the second electrode 202 and the third electrode 301 may be patterned from the same layer of the conductive film. The first electrode 201, the second electrode 202, the third electrode 301, and the fourth electrode 302 may each be a transparent electrode such as Indium Tin Oxide (ITO), but not limited thereto. In the present embodiment, the fourth electrode 302 has to be a transparent electrode so as to display a color changing phenomenon, and the specific materials of the other electrodes are not limited. On the basis of this, since the second electrode 202 and the third electrode 301 are disposed in the same layer, the bottom surface of the electrochromic module 30 is higher than the top surface of the piezoelectric module 20, so in the embodiment, a resin layer 60 may also be disposed between the flexible substrate 10 and the electrochromic modules 30 for adjusting the gap therebetween.

In the present exemplary embodiment, both the first attachment layer 40 and the second attachment layer 50 can be used as the attachment surface to detect the pressure change of the surface of the test subject, so both may have good viscoelastic properties and high sensitivity suitable to be the sensing surface of the piezoelectric module 20.

Optionally, when the first attachment layer 40 is used as the attachment surface, as shown in FIG. 2, the pressure visualization device can be folded along a boundary between the piezoelectric module 20 and the electrochromic module 30 to adhere the folded flexible substrate 10 together through the second attachment layer 50. Then the first attachment layer 40 is attached to the surface of the test subject to sense the pressure change of the surface of the test subject and the sensed pressure is transmitted to the piezoelectric module 20, and further to the electrochromic module 30 to be presented.

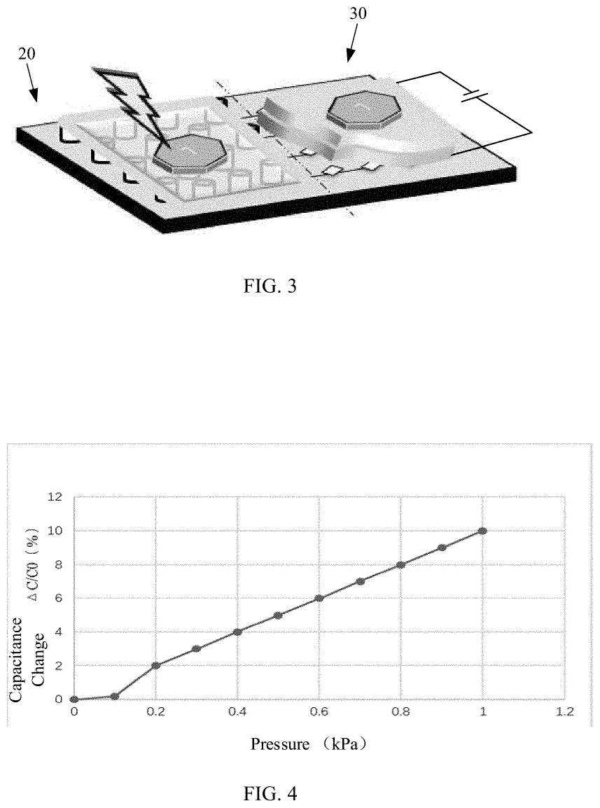

Optionally, when the second attachment layer 50 is used as the attachment surface, as shown in FIG. 3, the pressure visualization device can be directly attached with the second attachment layer 50 to the surface of the test subject to sense the pressure change of the surface of the test subject, and the sensed pressure is transmitted to the piezoelectric module 20, and further to the electrochromic module 30 to be presented.

It can be seen that the pressure visualization device has two use states as shown in FIG. 2 and FIG. 3, which can be used for medical detection such as electrocardiographic monitoring, as long as the pressure visualization device is attached to the heart of the detection subject, the heartbeat signal graph can appear immediately with detection of the heartbeat of the subject, so that the heartbeat pattern of the detection subject can be observed in real time. It should be noted that the pressure visualization device only needs a power source such as a lithium battery of about −6V as a basic working voltage for ensuring its normal operation.

It should be understood that the present disclosure is not limited thereto, and in some specific embodiments, one or more of the first attachment layer 40 and the second attachment layer 50 may also be omitted or other attachments or fixing device can be used in place of the first attachment layer 40 and/or the second attachment layer 50.

On the basis of this, considering the foldability of the pressure visualization device, the flexible substrate 10 can be made of flexible material such as polyimide (Polyimide, PI), polycarbonate (Polycarbonate, PC), polyethylene (Polyethylene, PE), and polyethylene terephthalate (PET). The length of the area occupied by the piezoelectric module 20 preferably does not exceed half of the total length of the flexible substrate 10, so as to facilitate the piezoelectric module 20 to be folded to the back of the electrochromic module 30. In this way, the pressure visualization device not only has a smaller volume when folded, but also since the first attachment layer 40 directly contacts the piezoelectric module 20, compared to disposing the flexible substrate 10 between the second attachment layer 50 and the piezoelectric module 20, the pressure visualization device can have a higher sensitivity.

Further, the materials of the first attachment layer 40 and the second attachment layer 50 may each be a hydrogel. Specifically, the present embodiment can form a hydrogel by physically cross linking Amorphous Calcium Carbonate (ACC) nanoparticles, Polyacrylic Acid (PAA), and sodium alginate. Among them, ACC has the properties of variability, plasticity, controllability, etc.; sodium alginate can rapidly form a gel under mild conditions; due to the presence of Ca²⁺ in ACC, Na⁺ on the G unit can undergo ion exchange reaction with divalent cations, and the G unit stack forms a crosslinked network structure to rapidly form a hydrogel; and PAA can form a stable compound with Ca²⁺ to make the structure of the hydrogel more stable.

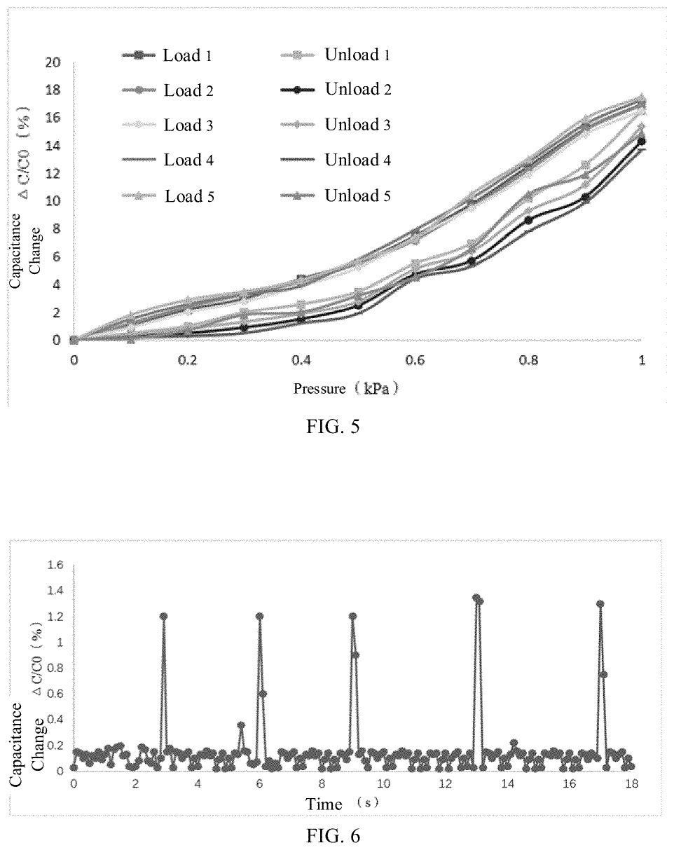

Based on this, on the one hand, the hydrogel prepared in this embodiment has a unique viscoelastic property, which can adhere the two folded parts together, and on the other hand, has good mechanical adaptability (including flexibility, stretchability and easy processing, completely self-repairing) and high sensitivity, as well as a high degree of matching and fitting effect on non-linear surfaces and dynamic surfaces, which can sense small changes in external pressure such as human motion or water droplets. On a further hand, the effect on the skin is small, and therefore, it is suitable for direct attachment to the skin surface. FIG. 4 schematically illustrates a capacitance-pressure response graph of a hydrogel pressure sensor in a pressure range of 0˜1 kPa. FIG. 5 schematically illustrates a capacitance-pressure cycle graph of a hydrogel pressure sensor. FIG. 6 schematically illustrates a real-time capacitance response graph of a hydrogel pressure sensor detecting water droplets. It can be seen that the hydrogel pressure sensor, that is, the piezoelectric module 20 with the hydrogel as the sensing surface, has high sensitivity and good repair performance.

In the present exemplary embodiment, the piezoelectric unit 200 of the piezoelectric module 20 may include at least a first electrode 201, a second electrode 202, and a piezoelectric layer 203. In order to improve the electrical conductivity of the piezoelectric module 20, a conductive layer 204 such as a gold conductive layer may be disposed between the first electrode 201 and the piezoelectric layer 203. The conductive layer 204 may include a plurality of independent conductive blocks, and the plurality of conductive blocks may be disposed in one-to-one correspondence with the plurality of piezoelectric layer units of the piezoelectric layer 203. The piezoelectric layer 203 may include a film layer including a piezoelectric material such as zinc oxide nanowire, graphene or carbon nanotube, wherein the zinc oxide nanowire has excellent electrical conductance transmission efficiency, light transmittance, and bacteriostasis.

In the present exemplary embodiment, the electrochromic units 300 of the electrochromic module 30 may include at least a third electrode 301, a fourth electrode 302, an electrochromic layer 303, and an ion transport layer 304. In order to protect the surface of the electrochromic module 30 from damage, a protective layer 305 such as a transparent resin layer may be disposed on a side of the fourth electrode 302 facing away from the flexible substrate 10, and specifically may be resin material such as polydimethylsiloxane (PDMS). In order not to affect the piezoelectric sensing effect, the protective layer 305 covers only the area where the electrochromic module 30 is located. The electrochromic layer 303 may include a pattern layer including an electrochromic material such as tungsten trioxide, polyaniline or a derivative thereof, wherein the electroluminescence property of the tungsten trioxide can exhibit good cycle stability. For example, it maintains more than 85% color contrast after 300 cycles. A current amplifying circuit may also be disposed in the electrochromic layer 303, for amplifying the minute current to drive the electrochromic layer 303 to emit light efficiently. It should be noted that the technology of the current amplifying circuit is relatively mature, and will not be described here.

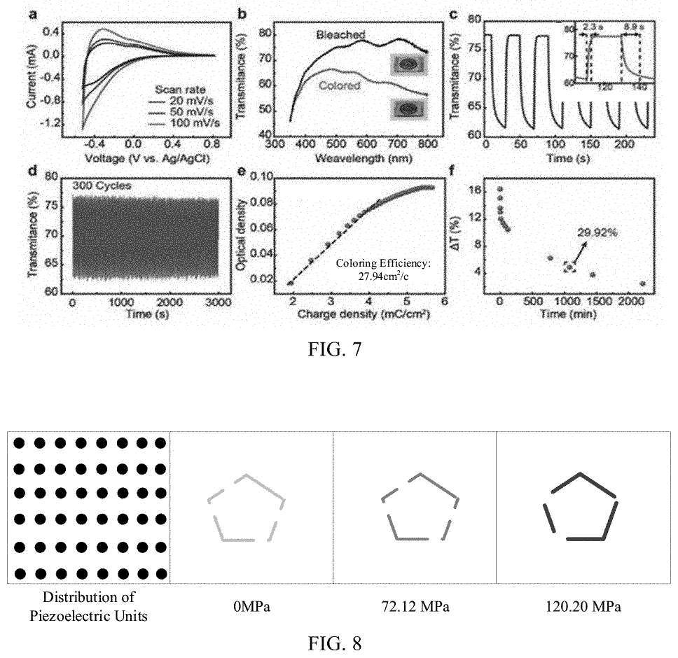

FIG. 7 shows a plurality of performance graphs of the electrochromic module 30 employing the electrochromic layer 303 of tungsten trioxide. Where, Figure a is the cyclic volt-ampere graph of the tungsten trioxide flakes, showing cyclic volt-ampere graphs of voltages between −0.5 and 0.8 V at scan rates of 20, 50 and 100 mV/s; Figure b is a UV-visible spectrum of a coloring process and a bleaching process of the electrochromic part at a bias voltage of −2V and +2V; Figure c is the color conversion behavior measured at a wavelength of 632.8 nm, where the inset is an enlarged view of a single conversion cycle; Figure d is a schematic diagram of cycle stability after more than 300 cycles; Figure e is a test chart of optical density and carrier density at a wavelength of 632.8 nm, with the coloring efficiency of 27.94 cm²/C; and Figure f is an effect diagram of a test on electrochromic color changing and color retention ability at transmittance of a wavelength of 632.8 nm.

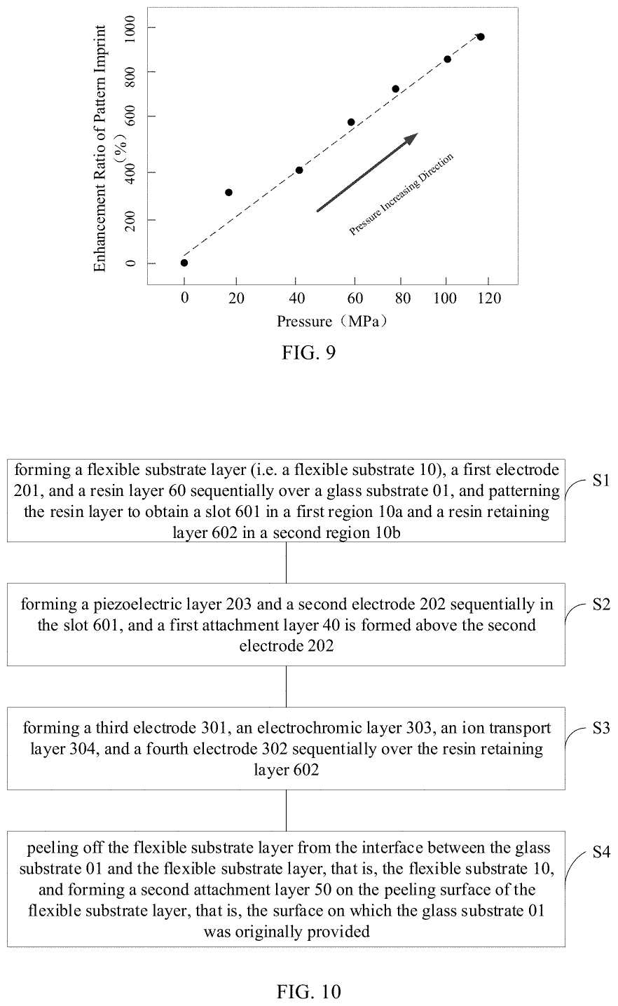

In the pressure visualization device provided by the exemplary embodiment of the present disclosure, each piezoelectric unit 200 of the piezoelectric module 20 is connected to the electrochromic unit 300 of the electrochromic module 30 correspondingly. In use, the hydrogel on either side may be attached to the surface of the detection subject. As the surface pressure of the detection subject changes, a current is generated in the piezoelectric module 20 due to the piezoelectric effect, and the higher the pressure, the larger the current is. The current is transmitted to the electrochromic module 30 and excites the electrochromic layer 303 to emit light and change color according to the position of the current. The path or pattern in which the pressure is generated at the piezoelectric layer 203 is recorded. If the pressure generated by the detection subject is small, the piezoelectric module 20 can only generate a weak current. At this time, the current amplifying circuit disposed in the electrochromic layer 303 enlarges the weak current, and the amplified current is sufficient to excite the electrochromic layer 303 to emit light and change color. The path or pattern in which the pressure is generated at the piezoelectric layer 203 is recorded. For example, when a partial pressure, such as a pentagon pressure, is applied to the surface of the piezoelectric module 20, it can generate a piezoelectric polarization charge at the edge of the piezoelectric module 20, thereby causing current transported in the system, which is finally presented by color changing of the electrochromic module 30. FIG. 8 is a diagram showing a distribution effect of the piezoelectric units 200 in the piezoelectric module 20 and pattern imprints displayed by the electrochromic module 30 under different pressures, for example, a pattern imprint of the pressure generated by a pentagonal object displayed after current amplification. FIG. 9 is a view showing a linear relationship between the enhancement ratio of the pattern imprint and the applied pressure, for example, a linear graph in which the pentagon pattern imprint is increased from 0 to 900% when the applied pressure is increased from 0 to 120.20 MPa. It should be understood that the linear relationship of FIG. 9 is merely an example. In practical applications, other functional relationships may also be satisfied between the enhancement ratio of the pattern imprint and the applied pressure depending on the specifically employed structure and/or material.

The exemplary embodiment also provides a manufacturing method of a pressure visualization device that can be used to manufacture the pressure visualization device described above. As shown in FIG. 10, the manufacturing method of the pressure visualization device may include the following steps.

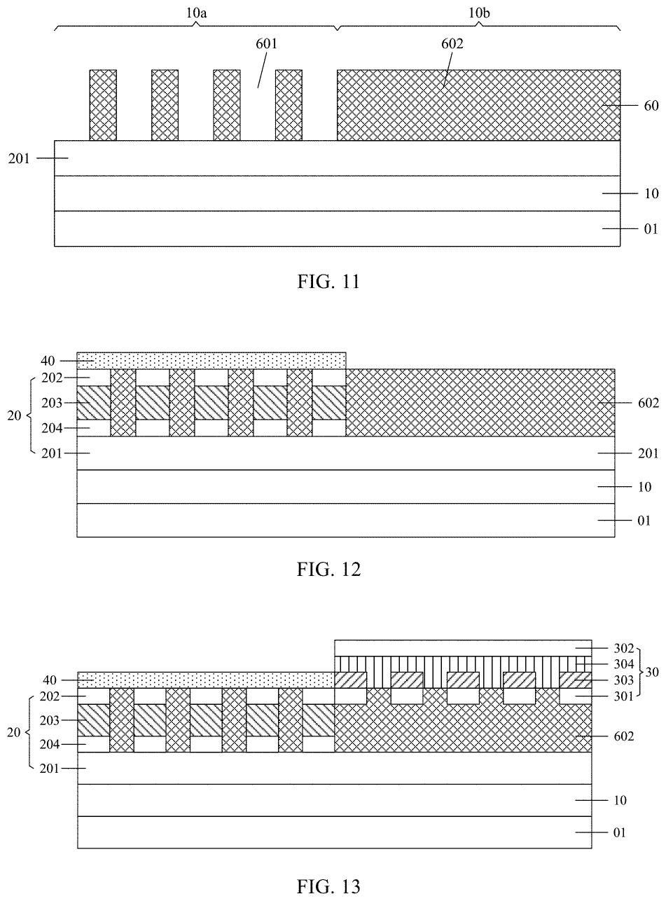

In S1, as shown in FIG. 11, a flexible substrate layer (i.e. a flexible substrate 10), a first electrode 201, and a resin layer 60 are sequentially formed over a glass substrate 01, and the resin layer is patterned to obtain a slot 601 in a first region 10a and a resin retaining layer 602 in a second region 10b.

The first region 10a and the second region 10b are disposed adjacent to each other, and the first electrode 201 may be laid on the surface of the entire flexible substrate 10 or only in the first region 10a.

In S2, as shown in FIG. 12, a piezoelectric layer 203 and a second electrode 202 are sequentially formed in the slot 601, and a first attachment layer 40 is formed above the second electrode 202.

Wherein, the second electrode 202 may include a plurality of independent electrode blocks, and the piezoelectric layer 203 may include a plurality of independent piezoelectric layer units corresponding to the plurality of electrode blocks of the second electrode 202.

In S3, as shown in FIG. 13, a third electrode 301, an electrochromic layer 303, an ion transport layer 304, and a fourth electrode 302 are sequentially formed over the resin retaining layer 602.

The second electrode 202 and the third electrode 301 are electrically connected to each other, and the third electrode 301 may include a plurality of independent electrode blocks, and the fourth electrode 302 may be a transparent plate electrode or a plurality of electrically connected transparent block electrodes. The electrochromic layer 303 may include a plurality of independent electrochromic layer units corresponding to the plurality of electrode blocks of the third electrode 301, and the ion transport layer 304 may include a full layer of ion transport layer or include a plurality of independent ion transport layer units.

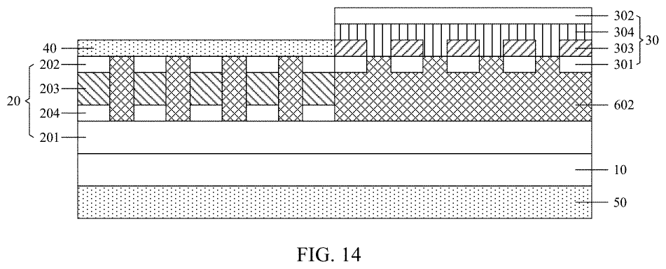

In S4, as shown in FIG. 14, the flexible substrate layer is peeled off from the interface between the glass substrate 01 and the flexible substrate layer, that is, the flexible substrate 10, and a second attachment layer 50 is formed on the peeling surface of the flexible substrate layer, that is, the surface on which the glass substrate 01 was originally provided.

The first attachment layer 40 and the second attachment layer 50 may be attached to the surface of the test subject for sensing the pressure change of the surface of the test subject.

Based on this, the first region 10a may be used to provide the piezoelectric module 20, and the piezoelectric module 20 may include a plurality of piezoelectric units 200 each including the first electrode 201, the second electrode 202, and the piezoelectric layer 203 between the first electrode 201 and the second electrode 202. The second region 10b may be used to provide the electrochromic module 30. The electrochromic module 30 may include a plurality of electrochromic layers 300 each including the third electrode 301, the fourth electrode 302, and the electrochromic layer 303 and the ion transport layer 304 between the third electrode 301 and the fourth electrode 302.

In the manufacturing method of a pressure visualization device provided by an exemplary embodiment of the present disclosure, by forming a piezoelectric module 20 and an electrochromic module 30 on one side of a flexible substrate 10, and a first attachment layer 40 outside the piezoelectric module 20, and forming a second attachment layer 50 on the other side of the flexible substrate 10 while maintaining an electrical connection between the piezoelectric module 20 and the electrochromic module 30, so that the piezoelectric module 20 can be convert the sensed pressure signal into an electrical signal, and then to excite the electrochromic module 30 to emit light and change color under the control of the electrical signal. It can not only realize detection of pressure, but also the detected pressure can be visually displayed instantly. It can be seen that the pressure visualization device can instantly display the pressure signal graph without an external display device, thereby realizing the visualization of the pressure signal. In addition, since the piezoelectric module 20 and the electrochromic module 30 are integrally disposed on the flexible substrate 10, the pressure visualization device also has the advantage of being small and portable.

In the present exemplary embodiment, the sum of the occupied area of the piezoelectric module 20 on the flexible substrate 10 (i.e. the area of the first region 10a) and the occupied area of the electrochromic module 30 on the flexible substrate 10 (i.e. the area of the second region 10b) may be equal to the surface area of the flexible substrate 10. The area of the first region 10a and the area of the second region 10b can be, for example, exactly the same.

The manufacturing method of the pressure visualization device provided by the exemplary embodiment will be specifically described below with reference to the accompanying drawings.

In step S1, the flexible substrate layer (i.e. the flexible substrate 10), the first electrode 201, and the resin layer 60 are sequentially formed over the glass substrate 01, and the resin layer is patterned to obtain the slot 601 at the first region 10a and the resin retaining layer 602 at the second region 10b.

The flexible substrate layer can be made of material such as Polyimide (PI), Polycarbonate (PC), Polyethylene (PE), and Polyethylene terephthalate (PET). The first electrode 201 may be an ITO electrode, and the resin layer 60 may be, for example, an SU-8 negative photoresist, which is suitable for preparing a microstructure having a relatively high depth-width ratio.

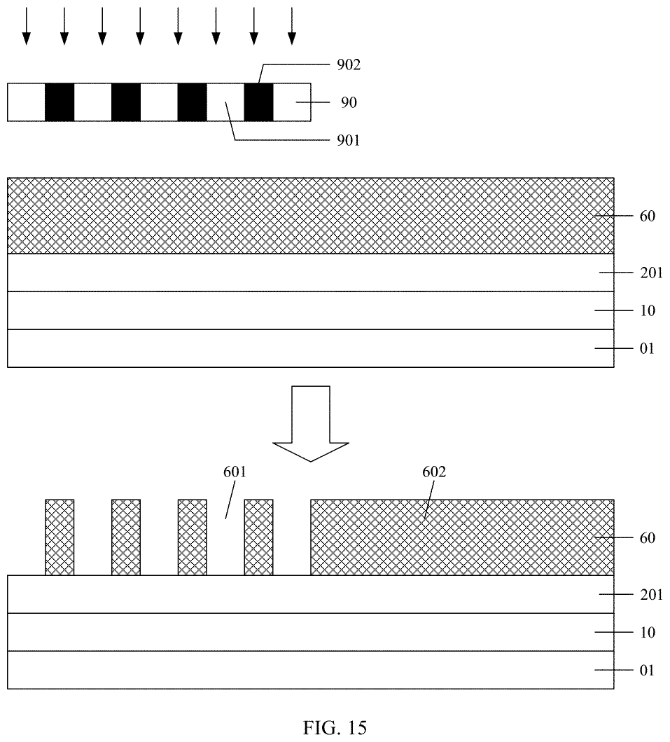

For example, as shown in FIG. 15, in this step, the flexible substrate layer such as a PI layer and the first electrode 201 such as an ITO layer may be sequentially formed over the glass substrate 01, and then the resin layer 60 such as a SU-8 negative photoresist is formed over the first electrode 201 by a coating process, and the slot 601 for accommodating the piezoelectric module 20 is prepared on one side of the resin layer 60, for example, the left side region. The actual area of the left region can be determined according to different needs, with a length preferably not exceeding half of the total length of the flexible substrate 10 to facilitate folding. The process of forming the slot 601 can expose the resin layer 60 through the mask 90 and develop the exposed resin layer 60, thereby obtaining the slot 601 corresponding to the transparent region 901 of the mask and a resin retaining layer corresponding to the non-transparent region 902 of the mask. The slot 601 penetrates the resin layer 60 to a depth sufficient to prepare subsequent patterned layers such as the piezoelectric layer 203 and the second electrode 202.

In step S2, the piezoelectric layer 203 and the second electrode 202 are sequentially formed in the slot 601, and the first attachment layer 40 is formed above the second electrode 202.

The piezoelectric layer 203 may be, for example, a thin film formed of zinc oxide nanowires. The second electrode 202 may be, for example, a plurality of ITO electrode blocks. The first attachment layer 40 may be, for example, a hydrogel. Before forming the piezoelectric layer 203, a conductive layer 204 such as a gold conductive layer may be formed over the first electrode 201 in the slot 601, and the conductive layer 204 may include a plurality of independent conductive blocks corresponding to the plurality of electrode blocks of the second electrode 202.

It should be noted that electrical connection is required between the second electrode 202 and the third electrode 301, and the same material may be used for both. Therefore, the second electrode 202 and the third electrode 301 may be prepared separately or simultaneously. In view of the simplification of the manufacturing process, in the present embodiment, preferably, the second electrode 202 and the third electrode 301 are formed by one patterning process.

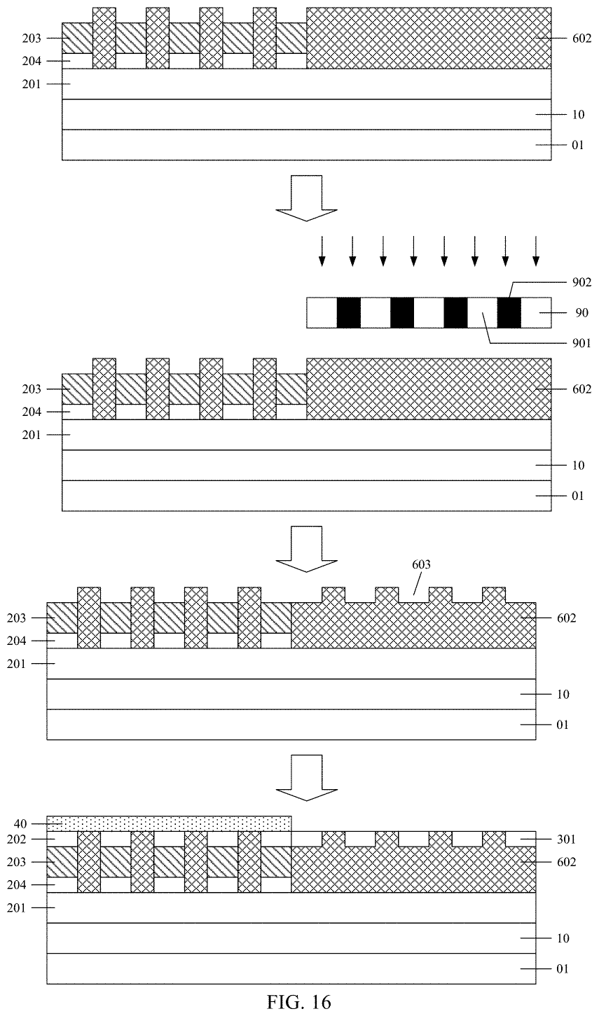

For example, as shown in FIG. 16, in this step, the conductive layer 204 such as a gold conductive layer and the piezoelectric layer 203 such as a zinc oxide nanowire film layer may be sequentially formed over the first electrode 201 exposed by the slot 601. Then, a recess 603 for accommodating the third electrode 301 of the electrochromic module 30 is prepared at the other side of the resin layer 60, for example, the right side region. Specifically, the resin retaining layer 602 at the right side region is exposed through a mask 90 and the resin retaining layer 602 after exposure is developed to obtain the recess 603 corresponding to the transparent region 901 of the mask. The lower surface of the recess 603 may be flush with the upper surface of the piezoelectric layer 203. On the basis of this, an electrode layer such as an ITO electrode layer is prepared on the surface of the entire substrate, wherein the electrode formed corresponding to the left side region is the second electrode 202, and the electrode formed corresponding to the right side region is the third electrode 301. Finally, a first attachment layer 40 such as a hydrogel is formed over the second electrode 202, thereby completing the manufacturing of the piezoelectric module 20.

The hydrogel can be obtained by physically cross linking ACC nanoparticles, PAA and sodium alginate. Among them, ACC has the properties of variability, plasticity, controllability, etc.; sodium alginate can rapidly form a gel under mild conditions; due to the presence of Ca²⁺ in ACC, Na⁺ on the G unit can undergo ion exchange reaction with divalent cations, and the G unit stack forms a crosslinked network structure to rapidly form a hydrogel; and PAA can form a stable compound with Ca′ to make the structure of the hydrogel more stable. It should be noted that the advantages of the hydrogel as the material of the attachment layer are described in detail above, and therefore will not be described herein.

In step S3, the third electrode 301, the electrochromic layer 303, the ion transport layer 304, and the fourth electrode 302 are sequentially formed over the resin retaining layer 602.

The third electrode 301 may be, for example, a plurality of ITO electrode blocks, and the fourth electrode 302 may be, for example, an ITO plate electrode or a plurality of electrically connected ITO block electrodes. The electrochromic layer 303 may be, for example, a tungsten trioxide pattern layer corresponding to the plurality of electrode blocks of the third electrode 301, and a current amplifying circuit may be formed in the tungsten trioxide pattern layer. The ion transport layer 304 may be, for example, a whole layer of ion transport layer 304 containing lithium ions Li⁺ or a plurality of independent ion transport layer units. A protective layer 305 such as a PDMS resin layer may also be formed over the fourth electrode 302 in consideration of the protection of the electrode.

It should be noted that since the third electrode 301 and the second electrode 202 can be formed at the same time, and the forming process thereof has been described in detail in the previous step, this step is not described in detail for the formation process of the third electrode 301. Nevertheless, the third electrode 301 may not be formed simultaneously with the second electrode 202. For example, only the second electrode 202 is formed in the previous step, and the third electrode 302 is formed in this step.

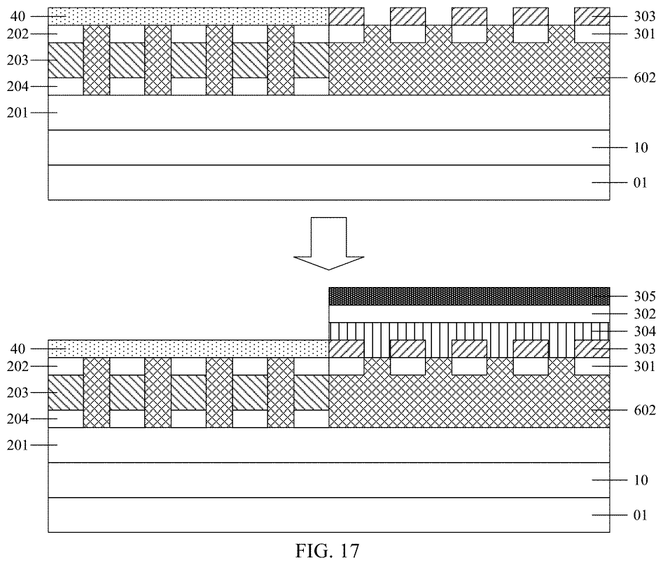

For example, as shown in FIG. 17, in this step, the electrochromic layer 303 such as a tungsten trioxide pattern layer, the ion transport layer 304 such as the whole layer of ion transport layer 304 containing lithium ions Li⁺, the fourth electrode 302 such as an ITO electrode and the protective layer 305 such as a PDMS resin layer may be sequentially formed over the third electrode 301, such as an ITO electrode, to complete the manufacturing of the electrochromic module 30. Here, a current amplifying circuit may be formed inside the electrochromic layer 303 such as the tungsten trioxide pattern layer for amplifying the minute current to drive the electrochromic layer 303 to perform effective light emission. It should be noted that the technology of the current amplifying circuit is relatively mature, and will not be described here.

In step S4, the flexible substrate layer is peeled off from the interface of the glass substrate 01 and the flexible substrate layer, that is, the flexible substrate 10, and the second attachment layer 50 is formed on the other side of the flexible substrate layer.

The second attachment layer 50 may be, for example, a hydrogel, and the hydrogel and the hydrogel used in the first attachment layer 40 may have the same composition, which can be obtained by physically cross linking ACC nanoparticles, PAA and sodium alginate.

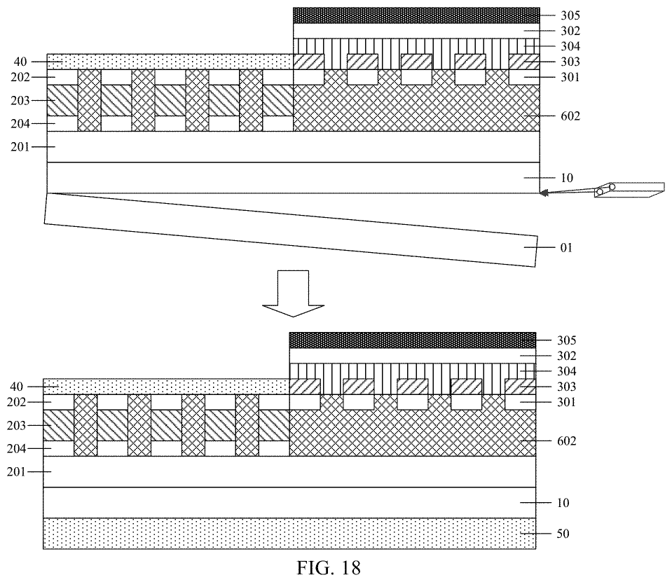

For example, as shown in FIG. 18, in this step, the glass substrate 01 may be peeled off from the flexible substrate 10 with a laser lift-off technique, and then a second attachment layer 50 such as a hydrogel is formed under the flexible substrate 10 to complete the manufacturing of the pressure visualization device.

In the pressure visualization device obtained by the above method, the hydrogel on either side may be attached to the surface of the detection subject. As the surface pressure of the detection subject changes, a current is generated in the piezoelectric module 20 due to the piezoelectric effect, and the higher the pressure, the larger the current is. The current is transmitted to the electrochromic module 30 and excites the electrochromic layer 303 to emit light according to the position of the current. The path or pattern in which the pressure is generated at the piezoelectric layer 203 is recorded. If the pressure generated by the detection subject is small, the piezoelectric module 20 can only generate a weak current. At this time, the current amplifying circuit disposed in the electrochromic layer 303 enlarges the weak current, and the amplified current is sufficient to excite the electrochromic layer 303 to emit light. The path or pattern in which the pressure is generated at the piezoelectric layer 203 is recorded.

It should be noted that the manufacturing method of the pressure visualization device can be adjusted according to actual conditions, but it should be within the protection scope of the present disclosure as long as the pressure visualization device provided by the exemplary embodiment can be formed.

The present example embodiment also provides a detection device including the above-described pressure visualization device, which may be a medical detection device such as a sphygmomanometer or an electrocardiograph. However, the detection device can also be applied to other fields than the medical field, which is not specifically limited in this embodiment. On the basis of this, considering the portability of the detection device, based on the structure of the pressure visualization device, it can be provided as a wearable device by adding a corresponding wearing connector, thereby facilitating ready use of medical devices such as an electrocardiograph.

It should be noted that although several modules or units of device for action execution are mentioned in the detailed description above, such division is not mandatory. Indeed, in accordance with embodiments of the present disclosure, the features and functions of two or more modules or units described above may be embodied in one module or unit. Conversely, the features and functions of one of the modules or units described above may be further divided into multiple modules or units.

In addition, although the various steps of the method of the present disclosure are described in a particular order in the drawings, this is not required or implied that the steps must be performed in the specific order, or all the steps shown must be performed to achieve the desired result. Additionally or alternatively, certain steps may be omitted, multiple steps may be combined into one step, and/or one step may be decomposed into multiple steps and the like.

Other embodiments of the disclosure will be apparent to those skilled in the art from consideration of the specification and practice of the disclosure disclosed here. This application is intended to cover any variations, uses, or adaptations of the disclosure following the general principles thereof and including such departures from the present disclosure as come within known or customary practice in the art. It is intended that the specification and examples be considered as exemplary only, with a true scope and spirit of the disclosure being indicated by the following claims.

It will be appreciated that the present disclosure is not limited to the exact construction that has been described above and illustrated in the accompanying drawings, and that various modifications and changes may be made without departing from the scope thereof. It is intended that the scope of the disclosure only be limited by the appended claims.

1. A pressure visualization device, comprising a flexible substrate, a piezoelectric module and an electrochromic module on a first surface of the flexible substrate;
wherein the piezoelectric module comprises a plurality of piezoelectric units each comprising a first electrode close to the flexible substrate, a second electrode away from the flexible substrate, and a piezoelectric layer between the first electrode and the second electrode;
the electrochromic module comprises a plurality of electrochromic units each comprising a third electrode close to the flexible substrate, a fourth electrode away from the flexible substrate, and an electrochromic layer between the third electrode and the fourth electrode; and
the second electrode is electrically connected to the third electrode, and the fourth electrode is a transparent electrode.

2. The pressure visualization device of claim 1, wherein a sum of an occupied area of the piezoelectric module on the flexible substrate and an occupied area of the electrochromic module on the flexible substrate is equal to a surface area of the flexible substrate.

3. The pressure visualization device of claim 1, further comprising a first attachment layer on a surface of the piezoelectric module facing away from the flexible substrate, a second attachment layer on a second surface of the flexible substrate, and the first attachment layer and the second attachment layer each comprises a hydrogel.

4. The pressure visualization device of claim 3, wherein the hydrogel is formed by physical crosslinking of amorphous calcium carbonate nanoparticles, polyacrylic acid, and sodium alginate.

5. The pressure visualization device of claim 1, wherein the second electrode and the third electrode are disposed in a same layer and comprise a same material.

6. The pressure visualization device of claim 1, further comprising a protective layer on a side of the electrochromic module facing away from the flexible substrate.

7. The pressure visualization device of claim 6, wherein the protective layer comprises a transparent resin layer and the transparent resin layer comprises polydimethylsiloxane.

8. The pressure visualization device according to claim 1, wherein the piezoelectric module further comprises a conductive layer between the first electrode and the piezoelectric layer, and the piezoelectric layer comprises zinc oxide nanowires.

9. The pressure visualization device according to claim 1, wherein the electrochromic layer comprises a tungsten trioxide pattern layer, and a current amplification circuit is provided in the tungsten trioxide pattern layer.

10. A manufacturing method of a pressure visualization device, comprising:
forming a flexible substrate layer, a first electrode, and a resin layer sequentially over a glass substrate, and patterning the resin layer to obtain a slot in a first region and a resin retaining layer in a second region, the first region and the second region being disposed adjacent to each other;
forming a piezoelectric layer and a second electrode sequentially in the slot;
forming a third electrode, an electrochromic layer, and a fourth electrode sequentially over the resin retaining layer, the third electrode being electrically connected to the second electrode, and the fourth electrode being a transparent electrode; and
peeling off the flexible substrate layer from an interface of the glass substrate and the flexible substrate layer;
wherein the first region is configured to form a piezoelectric module, the piezoelectric module comprises a plurality of piezoelectric units each comprising the first electrode, the second electrode, and the piezoelectric layer; the second region is configured to form an electrochromic module, and the electrochromic module comprises a plurality of electrochromic units each comprising the third electrode, the fourth electrode, and the electrochromic layer.

11. The manufacturing method according to claim 10, wherein a sum of an area of the first region and an area of the second region is equal to a surface area of the flexible substrate layer.

12. The manufacturing method according to claim 10, further comprising:
after sequentially forming the piezoelectric layer and the second electrode in the slot, forming a first attachment layer over the second electrode; and
after peeling off the flexible substrate layer from the interface between the glass substrate and the flexible substrate layer, forming a second attachment layer on a peeling surface of the flexible substrate layer,
wherein the first attachment layer and the second attachment layer each comprises a hydrogel.

13. The manufacturing method according to claim 12, wherein the hydrogel is formed by physical crosslinking of amorphous calcium carbonate nanoparticles, polyacrylic acid, and sodium alginate.

14. The manufacturing method according to claim 10, wherein the second electrode and the third electrode are formed from a same film layer in a same patterning process; and
before forming the second electrode and the third electrode, the manufacturing method further comprises:
patterning the resin retaining layer to obtain a recess for forming the third electrode.

15. The manufacturing method according to claim 10, further comprising:
forming a protective layer over the fourth electrode-, wherein the protective layer comprises a transparent resin layer, and the transparent resin layer comprises polydimethylsiloxane.

16. (canceled)

17. The manufacturing method according to claim 10, wherein the piezoelectric module further comprises a conductive layer formed between the first electrode and the piezoelectric layer, and the piezoelectric layer comprises zinc oxide nanowires.

18. The manufacturing method according to claim 10, wherein the electrochromic layer comprises a tungsten trioxide pattern layer, and a current amplifying circuit is further formed in the tungsten trioxide pattern layer.

19. A detection device, comprising the pressure visualization device of claim 1.

20. The detection device according to claim 19, comprising a sphygmomanometer or an electrocardiograph.

21. The detection device according to claim 19, wherein the detection device is a wearable device.
